(19) United States
(12) Patent Application Publication
(10) Pub. No.: US 2018/0200208 A1
(43) Pub. Date: Jul. 19, 2018

(54) COMPOSITIONS FOR IMPROVING OR MAINTAINING MYOCARDIAL FUNCTION

(71) Applicant: RepoCeuticals ApS, Hørsholm (DK)

(72) Inventor: Lars Otto Uttenthal, Madrid (ES)

(21) Appl. No.: 15/743,260

(22) PCT Filed: Jul. 19, 2016
(86) PCT No.: PCT/EP2016/067115

§ 371 Date: Jan. 9, 2018

(30) Foreign Application Priority Data
Jul. 20, 2015 (DK) PA201570480

(51) Int. Cl. A61K 31/165; A61K 31/122; A61K 31/385; A61K 9/00; A61P 9/10; A61P 9/00

(52) U.S. Cl. CPC A61K 31/165; A61K 31/122; A61P 9/00; A61K 9/0053; A61P 9/10; A61K 31/385

(57) ABSTRACT

Compositions comprising melatonin or derivatives thereof together with coenzyme Q10 and/or alpha-lipoic are provided for oral administration to act as a cardioprotective treatment for heart failure, myocardial ischemia, cardiomyopathies, cardiotoxicity, and in relation to cardiac interventions.

### FIELD OF INVENTION

The present invention provides compositions comprising combinations of antioxidant substances to counteract defects of myocardial metabolism that play a negative role in disorders such as heart failure, myocardial ischemia, cardiomyopathies, cardiotoxicity, and in the outcome of cardiac or coronary artery surgery or percutaneous coronary intervention. As such, it is relevant to the fields of cardiology, cardiac surgery and to general internal medicine.

### BACKGROUND OF THE INVENTION

### Pathological Mechanisms in Heart Failure

In heart failure, whether associated with ischemic heart disease, cardiomyopathy or cardiotoxicity, the energy supply to the surviving cardiomyocytes is defective. The energy is supplied by oxidative phosphorylation occurring in the mitochondria of the cardiomyocytes, and therapeutic measures directed at the cardiomyocytes themselves attempt to improve the impaired mitochondrial function. Formal myocardial ischemia-reperfusion injury may take place when a coronary artery is obstructed, e.g. by thrombus, and the obstruction is relieved by operative intervention. Similarly, formal myocardial ischemia-reperfusion injury may occur during the procedures of cardiac surgery. However, in chronic ischemic heart disease and other conditions associated with heart failure, ischemia and reoxygenation are partial processes that coexist in parallel and combine to interfere with adequate mitochondrial function. Nevertheless, the pathology of myocardial ischemia-reperfusion injury is illustrative of many of the processes that lead to defective mitochondrial function in many of the conditions associated with heart failure.

Myocardial ischemia-reperfusion injury is characterized by intracellular ion accumulation, resulting especially in intracellular calcium overload, which leads to myofibrillar hyper-contractility, ATP depletion, ultrastructural damage to mitochondria and myocardial stunning. The mitochondria may undergo permeability transition through the formation of mitochondrial permeability transition pores (mptps). These are voltage-dependent channels that are triggered by calcium overload and oxidative stress. The formation of mptps results in loss of the electrochemical gradient, uncoupling of oxidative phosphorylation with reduced ATP production and diversion of electron transport to the increased generation of free-radical reactive oxygen species (ROS) such as superoxide anions and hydroxyl radicals, which are released into the cytoplasm. The mitochondrial enzyme manganese superoxide dismutase (MnSOD), which is important for eliminating the superoxide anions generated in the mitochondria is itself partially destroyed. The excessive production of ROS can quickly overwhelm the cell's endogenous free radical scavenging system, and this triggers cellular injury by reaction of the ROS with lipids, proteins, and nucleic acids. In addition to damaging nuclear and cytosolic structures, the ROS can trigger the opening of the mptps resulting in a positive feedback loop of additional free radical release from the mitochondria, known as “ROS-induced ROS release”. Expression of inducible nitric oxide synthase (iNOS) is induced in the cardiomyocytes, which may lead to an overproduction of nitric oxide (NO) and the production of free-radical reactive nitrogen species (RNS) such as peroxynitrite, formed by the reaction of NO with superoxide anions. Peroxynitrite reacts strongly with tyrosine residues in the cellular proteins to form nitrotyrosine, thus impairing protein function.

In addition to the above, which may be regarded as a principal pathological mechanism affecting mitochondrial function in heart-failure-associated cardiac disorders, other mechanisms come into play that contribute to myocardial dysfunction. For example, endothelial activation and injury increase vascular permeability and the recruitment of inflammatory cells which invade the myocardium. These cells, particularly the neutrophils, may be directly toxic to the myocardium by secreting proteases, generating ROS, and occluding the microvasculature. The renin-angiotensin system is activated, and its end-product, angiotensin II, increases intracellular calcium levels in cardiomyocytes and smooth muscle cells, leading to positive inotropism, impairment of diastolic function and coronary vasoconstriction. Angiotensin II is itself cardiotoxic via the induction of tumor necrosis factor-alpha (TNF-α) and may lead to myocyte necrosis. TNF-α, interleukin (IL)-1β and IL-6, are important pathological factors in inflammatory responses during the pathological progression of myocardial failure and hypertrophy. They are released during chronic inflammation, either in endothelial cells or in cardiomyocytes, and inhibit electron transport through the mitochondrial complex I and complex III-ubiquinone cycle, facilitating the generation of ROS. A positive feedback loop may occur between angiotensin II and TNF-α, in that the latter increases the expression of angiotensin converting enzyme and increases angiotensin II production, while the latter induces TNF-α in the cardiomyocytes.

### Prevalence of Heart Failure

Heart failure is a major public health issue, with a prevalence of over 5.8 million in the USA, and over 23 million worldwide. The lifetime risk of developing heart failure is one in five. While the age-adjusted incidence of heart failure may have plateaued, the condition still carries substantial morbidity and mortality, with a 5-year mortality of 45-60%, equivalent to that of many cancers. Heart failure represents a considerable burden to the health-care system, responsible for costs of more than $39 billion annually in the USA alone, and high rates of hospitalizations, readmissions, and outpatient visits.

### Medical Need for the Treatment of Heart Failure

There is at present no curative treatment for heart failure. Existing therapies are able to relieve symptoms but are unable to reverse the molecular changes that occur in the cardiomyocytes. There is clearly a need for therapies that are directed at counteracting the molecular pathology, perhaps particularly in relation to reversing or compensating for the changes leading to mitochondrial dysfunction.

In this respect, many synthetic pharmacological agents have been proposed that may reverse or block specific steps of the molecular pathology affecting the mitochondria. These have been studied in animal models without so far having made the transition to effective human therapy. However, the present invention focuses on a selection of molecules that are native to the human body (and may be present in all animals) and which have properties that can ameliorate the molecular pathology of the failing myocardium. These comprise melatonin and its antioxidant metabolites, derivatives or analogues, coenzyme Q10 and its analogues, and alpha-lipoic acid.

### SUMMARY OF THE INVENTION

In view of the above considerations, the invention consists of providing pharmaceutical compositions for oral administration comprising melatonin or an antioxidant metabolite, derivative or analogue thereof that does not materially interact with melatonin receptors, such as N¹-acetyl-N²-formyl-5-methoxykynuramine (AFMK), in combination with coenzyme Q10 and/or alpha-lipoic acid to moderate or reverse the progression of heart failure and adverse consequences of myocardial ischemia, cardiomyopathies, cardiotoxicity, and to act as a cardioprotective treatment before and/or after cardiac or coronary artery surgery or percutaneous coronary intervention. The advantage of the invention is that it provides new combinations of agents that reinforce each other's action to maintain adequate myocardial mitochondrial function and to scavenge the free radicals that are formed in excess in impaired mitochondrial function. When the compositions comprise an antioxidant metabolite of melatonin, this will not be subject to the reduction in bioavailability of oral melatonin due to first-pass metabolism in the liver. In respect of an antioxidant metabolite, derivative or analogue of melatonin that does not materially interact with melatonin receptors, the absence of material interaction is defined as being substantially lower than the binding affinity of melatonin to its transmembrane receptors MT1 and MT2 as further mentioned in the detailed description, such as at least 10-fold lower, for example at least 20-, 50-, or 100-fold lower (i.e. 10% or less, such as 5% or less, such as 2% or less, or 1% or less of the binding affinity of melatonin).

An optional feature of the invention is that a composition of the invention containing melatonin is administered only immediately before retirement for sleep, while a composition of the invention that has no known soporific effect is provided for use during waking hours.

Accordingly, the pharmaceutical compositions comprise essentially:
A composition comprising melatonin or an antioxidant metabolite, derivative or analogue thereof that does not materially interact with melatonin receptors, such as AFMK, in combination with coenzyme Q10 and/or alpha-lipoic acid, formulated to be suitable for oral administration, to act as a cardioprotective treatment for heart failure and adverse consequences of myocardial ischemia, cardiomyopathies, cardiotoxicity, and cardiac interventions.

For use during waking hours, any melatonin or substance with the soporific effect of melatonin is omitted and replaced with an alternative cardioprotective antioxidant, which may be a metabolite, derivate or analogue of melatonin that does not materially interact with melatonin receptors, such as AFMK, or a substance unrelated to melatonin, such as the polyphenols resveratrol, naringenin or a proanthocyanidin or mixture of proanthocyanidins.

The invention fulfills the medical need for a therapy directed at counteracting the molecular pathology of myocardial dysfunction in these conditions, particularly in relation to reversing or compensating for the changes leading to mitochondrial dysfunction.

In the following detailed description of the invention, details of the scope of the invention will be given, together with details of the practical performance of the invention.

### DETAILED DESCRIPTION OF THE INVENTION

### Active Ingredients

The principal active ingredient of the compositions of the invention is melatonin or an antioxidant metabolite, derivative or analogue thereof that does not materially interact with melatonin receptors, such as AFMK, in combination with the other antioxidants described below. In a diurnal medication schedule employing a composition containing melatonin itself, this composition will be administered only before retiring to sleep and not during waking hours.

### Melatonin

Melatonin (N-acetyl-5-methoxytryptamine) is a hormone produced by the pineal gland in human beings and other mammals by enzymatic modification of the amino acid tryptophan. Melatonin is involved in maintaining the circadian rhythm of various biological functions, being secreted in hours of darkness and acting on high-affinity melatonin Gi-coupled transmembrane receptors MT1 and MT2, which are widely distributed in many cells and tissues of the body. At the same time melatonin acts at supraphysiological concentrations as a powerful antioxidant and free radical scavenger for ROS and RNS (Gomez-Moreno et al 2010). It shows an especially high efficiency as a scavenger of hydroxyl radicals (Tan et al 1993). By preventing cardiolipin peroxidation by ROS, melatonin can inhibit the formation of mptps and potentially break the vicious circle of ROS-induced ROS release. Melatonin can also activate cytoprotective antioxidative enzymes such as copper-zinc superoxide dismutase (CuZnSOD) and the mitochondrial MnSOD, as well as glutathione peroxidase (Rodriguez et al 2004). In addition, melatonin also has anti-inflammatory effects to prevent the upregulation or cause the down-regulation of the expression of nuclear factor kappa B (NF-κB) and pro-inflammatory cytokines such as TNF-α and IL-1β. Melatonin readily penetrates cell membranes by a non-receptor-dependent mechanism and also crosses intracellular membranes. The highest intracellular concentrations of melatonin are found in the mitochondria, a feature that would favor its ameliorating effects on the molecular pathology of heart failure at the site where it is most needed. Patients with myocardial ischemia show lower endogenous melatonin concentrations at night, and circadian disruption results in increased risk for myocardial ischemia. These are indications that there are correlations between melatonin levels and myocardial ischemia even within the physiological range of these levels.

### Melatonin Metabolites, Derivatives and Analogues

The primary free-radical-scavenging and antioxidant activities of melatonin are not receptor-mediated. The receptor-mediated activities of melatonin, such as those involved in the maintenance of circadian rhythms and the corollary activity of having a mild soporific effect, are additional to its potential use as an antioxidant cardioprotective agent. The mild soporific effect of melatonin attributed to action on the MT1 receptor and is one of its best-studied activities. It is the subject of a meta-analysis of randomized controlled trials in patients with primary sleep disorders (Ferracioli-Oda et al 2013). Melatonin at oral doses of up to 5 mg reduces sleep latency and increases sleep duration by an average of 7-8 minutes, while also improving sleep quality by objective and subjective assessments. Higher doses of melatonin do not seem to increase the soporific effect above this subtle level (Zhdanova 2005). However, as there is no particular reason to wish for a cardioprotective agent that is inevitably accompanied by a soporific effect, however mild, it is of interest to seek melatonin-related compounds that retain the cardioprotective potential of the parent molecule, but show a strongly reduced or absent interaction with the melatonin receptors at their therapeutic levels. Many chemical derivatives of melatonin, including breakdown products and natural metabolites of melatonin, retain the antioxidant and free-radical-scavenging properties of the parent molecule. In this respect it is of interest to examine the antioxidant metabolites, derivatives and analogues of melatonin.

### Melatonin Metabolites.

In non-hepatic tissues, the reaction of melatonin with two hydroxyl radicals yields the metabolite cyclic 3-hydroxymelatonin (C3-OHM), which undergoes further oxidation by two hydroxyl radicals to break the indole ring and form N¹-acetyl-N²-formyl-5-methoxykynuramine (AFMK) (Tan et al 1993; Reiter et al 2007). C3-OHM is therefore also an effective antioxidant and hydroxyl radical scavenger. The reaction of melatonin with the hydroxyl radical precursor, hydrogen peroxide, similarly leads to production of AFMK. AFMK is also a reducing agent, capable of donating electrons to detoxify radical species, and has been shown to preserve the integrity DNA exposed to oxidizing agents. The action of aryl formamidase or catalase on AFMK produces N¹-acetyl-5-methoxykynuramine (AMK), which is an even more effective scavenger of hydroxyl radicals and reactive nitrogen species, protecting proteins from oxidative destruction. In this process, 3-acetamidomethyl-6-methoxycinnolinone (AMMC) or 3-nitro-AMK (AMNK) are formed. None of these metabolites of melatonin is known to bind to the melatonin receptors.

The liver is the principal site of the classically reported metabolic pathway for melatonin. This consists chiefly of 6-hydroxylation by the cytochromes P450 CYP1A1, CYP1A2, and CYP1B1, and the formation of the minor metabolite N-acetylserotonin by CYP2C19. The main product 6-hydroxymelatonin (6-OHM) is then conjugated at the hydroxyl group to form the 6-OHM glucuronide or 6-OHM sulfate. 6-OHM is an effective free radical scavenger in a variety of situations (Maharaj et al 2007), and shows a reduced affinity of 20% and 13% of that of melatonin for the recombinant MT1 and MT2 receptors, respectively (from data of Browning et al 2000).

N-acetylserotonin (NAS) is not only the immediate biosynthetic precursor but also a minor metabolite of melatonin. Like 6-OHM, it is conjugated to form the glucuronide or sulfate. Its protective effect against oxidative damage in certain model systems is reportedly 5 to 20 times as strong as that of melatonin (Oxenkrug 2005). Its affinities for recombinant MT1 and MT2 receptors are 0.14% and 0.22%, respectively, of those of melatonin (from data of Browning et al 2000). In the choice of a potential cardioprotective agent from the metabolites of melatonin, it may be of interest to avoid receptor-mediated melatonin effects.

### Melatonin Derivatives or Analogues.

Melatonin can also be chemically modified by introducing chemical groups at one or more of any of its constituent atoms susceptible of such modification or by introducing such groups in de novo synthesis of melatonin analogues or derivatives. Such modifications, which do not alter the fundamental indole structure of melatonin, are herein called derivatives. The fundamental indole structure of melatonin can also be modified by substituting other bicyclic aromatic structures. Such modifications are herein called analogues, which may also have different chemical side groups removed, introduced or modified. In the context of the present invention, the derivatives or analogues of melatonin of most interest are those that show neither marked agonist nor antagonist activity while retaining the membrane-penetrating, antioxidant and free-radical-scavenging capacities of melatonin.

### Coenzyme Q10 (Ubiquinone, Ubidecarenone)

Coenzyme Q10 is a 1,4-benzoquinone derivative, the number 10 referring to the number of isoprenyl chemical subunits in its side chain at position 3. It plays a central role in the mitochondrial respiratory chain through shuttling between three redox forms, ubiquinone, ubisemiquinone and ubiquinol. It acts as a carrier accepting electrons from mitochondrial complex I and complex II and transferring them to complex III. As such, it has been used as a nutritional supplement to support the failing myocardium. Coenzyme Q10 may help to reduce the production of mitochondrial ROS and reduce their toxic effects by its action as a free radical scavenger. Coenzyme Q10 may also have a role in stabilizing myocardial calcium-dependent ion channels and preventing the consumption of metabolites essential for adenosine-5′-triphosphate (ATP) synthesis. In a comprehensive review, Pagano et al (2014) reported 39 studies, 32 of them controlled, on the effects of coenzyme Q10 supplementation in a total of 3386 patients with cardiovascular diseases. The studies concern conditions that are only peripherally or indirectly relevant the long-term use of coenzyme Q10 to treat heart failure. In a meta-analysis restricted to randomized controlled trials of parallel or cross-over design in patients with heart failure (7 studies with 914 participants), Madmani et al (2014) were unable to draw a conclusion because of lack of information on clinically relevant endpoints.

### Coenzyme Q10 Derivatives or Analogues.

There are a number of derivatives or analogues of coenzyme Q10 that have similar antioxidant properties and may be used in substitution of the parent compound. Non-limiting examples of these are coenzyme Q9, which is also a natural cell constituent, the synthetic derivative decylubiquinone, and the analogue idebenone.

### Alpha-Lipoic Acid (Thioctic Acid)

Alpha-lipoic acid is an essential, normal constituent of cells and is chemically an organosulfur compound derived from octanoic acid. It is an essential cofactor in the citric acid cycle for mitochondrial α-ketoacid (e.g. pyruvate) dehydrogenase enzyme complex. Alpha-lipoic acid exists in two redox states, the reduced thiol form being a potent mitochondrial antioxidant, a metal chelator and a repletor of the reduced form of glutathione (GSH). It is an effective free radical scavenger. Pagano et al (2014) reported four controlled studies on the effects of alpha-lipoic acid supplementation in a total of 137 patients with cardiovascular diseases. These studies concern conditions that are only peripherally or indirectly relevant the long-term use of alpha-lipoic acid to treat heart failure. Specifically in heart failure, alpha-lipoic acid has shown an ameliorating effect on oxidative parameters in animal models. Acute administration to heart failure patients in an antioxidant cocktail with vitamins C and E before exercise produced an improvement in the cardiac index (Witman et al 2012), but longer-term effects were not studied.

### Additional Natural Antioxidants

### Vitamin E (Alpha-Tocopherol).

Vitamin E has antioxidant effects that are synergic with those of melatonin (see Reiter et al 2007). However, its long-term use as a single cardioprotective agent has paradoxically been associated with an increased incidence and exacerbation of heart failure. Its status in the combinations of antioxidants to achieve a cardioprotective effect is uncertain.

### Vitamin C (Ascorbic Acid).

This also has antioxidant effects that are synergic with those of melatonin (see Reiter et al 2007). Two large, independent studies have shown that plasma levels of vitamin C are inversely associated with the risk of heart failure in elderly men, but the use of vitamin C supplementation as a single cardioprotective agent has not been effective.

### Polyphenols.

Various polyphenols of plant origin have antioxidant and free-radical-scavenging properties, and have been shown to have cardioprotective effects in preclinical studies. These include resveratrol, which has been shown to induce mitochondrial MnSOD expression and activity (Robb et al 2008), naringenin, which like apigenin and chrysin is a 5-hydroxy flavone that reduces tissue injury and improves post-ischemic functional recovery in perfused rat hearts subjected to ischemia-reperfusion injury (Testai et al 2013), and a proanthocyanidin or mixture of proanthocyanidins from grape seed or other parts of the vine, which have shown similar cardioprotective effects.

### Choice and Characteristics of the Chosen Melatonin Metabolite:

Of the melatonin metabolites described above, N¹-acetyl-N²-formyl-5-methoxykynuramine (AFMK) is the preferred candidate for use on the basis of chemical stability and an expectancy of relative lack of adverse effects and specific actions unrelated to the purpose of the invention. Like melatonin, AFMK is a white powder that is insoluble in water but soluble in DMSO. It shows good long-term stability at 2-8° C.

### Choice and Characteristics of Antioxidant Melatonin Derivatives:

The chemical structure of melatonin can be represented as in Figure (I), in which sites suitable for chemical modification by the substitution of different chemical groups have been indicated by R₁, R₂, R₃, R₄, R₅ and R₆. These numbers do not correspond to the conventional numbering of positions in the indole ring of melatonin.

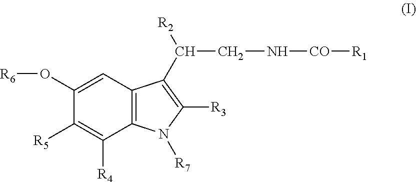

In native melatonin, R₁ and R₆ represent CH₃, while R₂, R₃, R₄, R₅ and R₇ represent H. Antioxidant melatonin derivatives with reduced binding to the melatonin receptors may comprise, as non-exclusive examples, those in which
R₁ represents H, a linear or branched C₁-C₄ alkyl group or a C₁-C₄ alkoxy group,
R₂ represents H or a linear or branched C₁-C₄ alkyl group,
R₃ represents H or a linear or branched C₁-C₄ alkyl group,
R₄ represents H
R₅ represents H or a linear or branched C₁-C₄ alkyl group,
R₆ represents H or a linear or branched C₁-C₄ alkyl group,
R₇ represents H, a linear or branched C₁-C₄ alkyl group, a —C(═O)—O—Rₐ group or a —C(═O)—N(H)—Rₐ group wherein Rₐ is a linear or branched C₁-C₄ alkyl group, the —CH₂—NH—C(═O)—R₁ side chain is extended by duplicating, triplicating or quadruplicating the —CH₂— group,
   or pharmaceutically acceptable salts of such derivatives.

These derivatives will show physicochemical properties similar to those of melatonin itself.

### Characteristics of Coenzyme Q10 and its Derivatives and Analogues:

Coenzyme Q10 is an almost water-insoluble solid of low melting point. It can be dissolved in ethanol, dimethyl sulfoxide and various oils. Non-limiting examples of antioxidant derivatives or analogues of coenzyme Q10 that may be used instead of coenzyme Q are the naturally occurring coenzyme Q9 or the synthetic derivative decylubiquinone, as well as the synthetic analogue idebenone. In water-based compositions, coenzyme Q10 and the other compounds mentioned can be rendered water-soluble by adsorption to a biologically acceptable carrier such as beta-cyclodextrin during the formulation process.

### Characteristics of Alpha-Lipoic Acid:

This is also referred to as R-(+)-alpha-lipoic acid or (R)-thioctic acid. It is an almost water-insoluble solid, but is soluble in ethanol, dimethyl sulfoxide and various oils. In water-based compositions, its sodium salt, sodium thioctate, can be used. This is soluble in water to yield solutions of near-physiological pH.

### Characteristics of Resveratrol, Naringenin and Proanthocyanidins:

These are antioxidant polyphenols that can be used to substitute melatonin in compositions of the invention intended for administration during waking hours. Like the other active ingredients mentioned, they are all almost insoluble in water, but readily soluble in ethanol, dimethyl sulfoxide and various oils. The proanthocyanidins form a large family of closely related polyphenols, being flavonoid oligomers of catechin, epicatechin and their gallic acid esters forming classes A (1, 2). B (1-8) and C (1-3). No definite distinction has been made between their antioxidant properties. As used herein, the term proanthocyanidin refers to any proanthocyanidin or a mixture of proanthocyanidins found in grape seed.

It is a feature of the present invention that while the above active ingredients may have shown only modest cardioprotective effects when used individually or pairwise, or, like melatonin, may not have been studied for a beneficial action in heart failure, the chosen combinations of active ingredients will show an additive or synergistic effect when used to treat heart failure, providing effective amelioration or slowing deterioration.

In a preferred embodiment, the compositions of the invention intended for administration during waking hours contain resveratrol in substitution for melatonin.

### Formulations

The pharmaceutical composition of the present invention may be in the form of a solution, emulsion or suspension in a pharmaceutically oil or in an aqueous preparation.

The formulation typically contains melatonin or antioxidant metabolite or derivative thereof at a concentration ranging from 1 mg to 200 mg per milliliter of the composition. Formulations containing coenzyme Q10 or a derivative or analogue thereof, and/or alpha-lipoic acid, will typically also contain said substances at a concentration ranging from 1 mg to 200 mg per milliliter of the composition. If the solubility of the active ingredients is not great enough to permit the recommended individual dose to be given in a single capsule of suitable size, the individual dose may be given as two or more capsules. Different formulations may be prepared to allow a specific formulation containing melatonin to be given at night, while a formulation without melatonin is given during the day. Said daytime formulation contains the non-melatonin ingredients at the concentrations stated above. In a preferred embodiment, resveratrol is substituted for melatonin in the formulation for daytime administration.

In a preferred embodiment of the formulation, the above active ingredients are dissolved in a pharmaceutically acceptable oil such as soybean oil, which is emulsified and contained in soft gelatin capsules of capacity up to 1 mL.

Formulations according to the present invention may comprise pharmaceutically acceptable carriers and excipients including microspheres, liposomes, micelles, microcapsules, nanoparticles or the like. For rendering the active ingredients soluble in aqueous media, excipients include water-soluble organic solvents such as polyethylene glycol 300, polyethylene glycol 400, ethanol, propylene glycol, glycerin, N-methyl-2-pyrrolidone, dimethylacetamide, and dimethyl sulfoxide, non-ionic surfactants such as Cremophor EL, Cremophor RH 40, Cremophor RH 60, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, and mono- and di-fatty acid esters of PEG 300, 400, or 1750. Water-insoluble lipids that may be used for oily preparations include castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil. For aqueous solutions or emulsions of water-insoluble compounds, various cyclodextrins such as alpha-cyclodextrin, beta-cyclodextrin, hydroxypropyl-beta-cyclodextrin, and sulfobutylether-beta-cyclodextrin can be used, as well as phospholipids such as hydrogenated soy phosphatidylcholine, distearoylphosphatidylglycerol, L-alpha-dimyristoylphosphatidylcholine, and L-alpha-dimyristoylphosphatidylglycerol. The chemical techniques to solubilize water-insoluble drugs for oral administration include pH adjustment, co-solvents, complexation, micro-emulsions, micelles, liposomes, and emulsions.

The use of these agents for preparing a pharmaceutically acceptable formulation of the active ingredients of the present invention are well-known to those of skill in the art, as are the methods of manufacturing suitable capsules, such as soft gelatin capsules for oral administration.

### Administration

Administration of the pharmaceutical composition is by swallowing the prescribed dose in the form in which it is presented, washed down with drinking water to ensure that the dose enters the stomach.

The pharmaceutical compositions may be presented in two readily distinguishable forms, one containing melatonin for administration immediately before retiring to sleep, referred to as nighttime administration, and the other, which does not contain melatonin, for administration during waking hours, referred to as daytime administration, the two compositions being intended to be used in conjunction.

### Indications

1. Heart failure due to any cause
2. Myocardial ischemia including chronic myocardial ischemia, unstable and stable angina, and recovery from myocardial infarction

### 3. Cardiomyopathies

4. Cardiotoxicity, e.g. due to anthracycline cytotoxic agents such as doxorubicin or platinum-based agents such as oxaliplatin
5. Preparation for and post-treatment of cardiac interventions such as cardiac or coronary artery surgery or percutaneous coronary intervention.

### Dose and Dosage Regimens

Due to the lack of generally agreed endpoints in clinical trials of the active ingredients, it is impossible to determine the “effective amount” of each of these ingredients. Instead, the doses used in clinical trials that have been declared to show a beneficial effect according to the various criteria of the authors provide a guide to doses of the compositions of the present invention.

These doses are not determined in relation to body weight or body surface area, but based on the dosage regimens used for treating adults in relevant clinical trials. They must be presumed to apply to adults with a body weight in the range of 45 kg to 130 kg, with a median body weight of 75 kg to 80 kg.

The standard single doses of melatonin, melatonin metabolite or derivative, co-enzyme Q10 or derivative, and alpha-lipoic acid are all in the range of 0.5 mg to 300 mg of each.

In the case of a formulation for daytime as opposed to nighttime administration that contains no melatonin, the doses of co-enzyme Q10 or a derivative thereof and alpha-lipoic acid, as well as any resveratrol or naringenin or proanthocyanidin added in substitution for melatonin, are in the range of 0.5 mg to 300 mg of each.

If the standard single dose of the compositions cannot, e.g. for reasons of solubility, be contained in a single capsule or other presentation for swallowing, the single dose can be administered as two or more units of presentation.

In a preferred embodiment, the composition for nighttime administration contains 20 mg of melatonin, 100 mg of co-enzyme Q10 and 100 mg of alpha-lipoic acid.

In a preferred embodiment, the composition for daytime administration contains 20 mg of resveratrol, 100 mg of co-enzyme Q10 and 100 mg of alpha-lipoic acid.

Each single dose of composition is preferably administered by being swallowed 1 to 4 times a day, preferably 3 times a day, before breakfast and lunch and before retiring at night.

The duration of dosing is determined by the attending physician and will typically range from 1 month to several years. Dosing may be started 2 weeks prior to a cardiac intervention and continued for 1 month or more after the intervention, at the discretion of the attending clinician.

### REFERENCES

Browning C, Beresford I, Fraser N, Giles H (2000) Pharmacological characterization of human recombinant melatonin mt(1) and MT(2) receptors. Br J Pharmacol 129:877-886.
Ferracioli-Oda E, Qawasmi A, Bloch M H (2013) Meta-analysis: melatonin for the treatment of primary sleep disorders. PLoS One 8:e63773.
Gomez-Moreno G, Guardia J, Ferrera M J, Cutando A, Reiter R J (2010) Melatonin in diseases of the oral cavity. Oral Dis 16:242-247.
Madmani M E, Yusuf Solaiman A, Tamr Agha K, Madmani Y, Shahrour Y, Essali A, Kadro W (2014) Coenzyme Q10 for heart failure. Cochrane Database Syst Rev 6:CD008684.
Maharaj D S, Glass B D, Daya S (2007) Melatonin: new places in therapy. Biosci Rep 27:299-320.
Oxenkrug G (2005) Antioxidant effects of N-acetylserotonin: possible mechanisms and clinical implications. Ann NY Acad Sci 1053:334-347.
Pagano G, Aiello Talamanca A, Castello G, Cordero M D, d'lschia M, Gadaleta M N, Pallardó F V, Petrovic S, Tiano L, Zatterale A (2014) Current experience in testing mitochondrial nutrients in disorders featuring oxidative stress and mitochondrial dysfunction: rational design of chemoprevention trials. Int J Mol Sci 15:20169-201208.
Reiter R J, Tan D X, Terron M P, Flores L J, Czarnocki Z (2007) Melatonin and its metabolites: new findings regarding their production and their radical scavenging actions. Acta Biochim Pol 54:1-9.
Robb E L, Page M M, Wiens B E, Stuart J A (2008) Molecular mechanisms of oxidative stress resistance induced by resveratrol: Specific and progressive induction of MnSOD. Biochem Biophys Res Commun 367:406-412.
Rodriguez C, Mayo J C, Sainz R M, Antolin I, Herrera F, Martin V, Reiter R J (2004) Regulation of antioxidant enzymes: a significant role for melatonin. J Pineal Res 36:1-9.
Tan D X, Chen L D, Poeggeler B, Manchester L C, Reiter R J (1993) Melatonin: a potent, endogenous hydroxyl radical scavenger. Endocrine J 1:57-60.
Testai L, Martelli A, Cristofaro M, Breschi M C, Calderone V (2013) Cardioprotective effects of different flavonoids against myocardial ischaemia/reperfusion injury in Langendorff-perfused rat hearts. J Pharm Pharmacol 65:750-756.
Witman M A, McDaniel J, Fjeldstad A S, Ives S J, Zhao J, Nativi J N, Stehlik J, Wray D W, Richardson R S (2012) A differing role of oxidative stress in the regulation of central and peripheral hemodynamics during exercise in heart failure. Am J Physiol Heart Circ Physiol 303:H1237-H1244.
Zhdanova I V (2005) Melatonin as a hypnotic: pro. Sleep Med Rev 9:51-65.

1. A composition comprising melatonin or an antioxidant metabolite, derivative or analogue thereof, in combination with coenzyme Q10 or an antioxidant derivative or analogue thereof, and/or alpha-lipoic acid.

2. The composition according to claim 1, in which the binding affinity of the antioxidant metabolite, derivative or analogue of melatonin to the melatonin transmembrane receptors is lower than the binding affinity of melatonin to said receptors, such as at least 10-fold lower, for example at least 20-, 50-, or 100-fold lower.

3. The composition according to claim 1, in which the antioxidant metabolite of melatonin is N¹-acetyl-N²-formyl-5-methoxykynuramine.

4. The composition according to claim 1, in which the antioxidant derivative or analogue of coenzyme Q10 is selected from the group consisting of coenzyme Q9, decylubiquinone and idebenone.

5. The composition according to claim 1, for daytime as opposed to nighttime administration, in which melatonin is substituted by resveratrol or naringenin or proanthocyanidin.

6. The composition according to claim 1 formulated for oral administration.

7. The composition according to claim 1 formulated as a medicament.

8. A method for treating a subject for heart condition, comprising administering a composition of claim 1 to the subject.

9. The method of claim 8, wherein the heart condition is myocardial ischemia.

10. The method of claim 8, wherein the composition is administered in the phase of recovery from myocardial infarction.

11. The method of claim 8, wherein the heart condition is a cardiomyopathy.

12. The method of claim 8, wherein the heart condition is cardiotoxicity, including cardiotoxicity due to an anthracycline cytotoxic agent such as doxorubicin or a platinum-based cytotoxic agent such as oxaliplatin.

13. The method of claim 8, wherein the composition is administered in the preparation for or post-treatment of cardiac interventions such as cardiac or coronary artery surgery or percutaneous coronary intervention.

14. The method of claim 8, wherein the composition is administered 1, 2, 3, or 4 times per day.

15. The method of claim 8, wherein the composition is administered over a period of 2 weeks or more, such as 1 month or more, such as 2 months or more, such as 3 months or more, such as 6 months or more, such as 1 year or more, such as 2 years of more, or as long as the patient has need of it.

16. The method of claim 8, wherein the single standard adult dose of melatonin or an antioxidant metabolite, derivative or analogue thereof is within the range of 0.5 mg to 300 mg.

17. The method of claim 8, wherein the single standard adult dose of coenzyme Q10 or an antioxidant derivative or analogue thereof is within the range of 0.5 mg to 300 mg.

18. The method of claim 8, wherein the single standard adult dose of alpha-lipoic acid is within the range of 0.5 mg to 300 mg.

19. The method of claim 8, wherein the daily dose of the composition is from one to four times the single dose of 0.5 mg to 300 mg.

20. The method of claim 8, wherein the composition is administered during the daytime or nighttime, wherein any melatonin is omitted from the composition for daytime administration.
